(19) United States
(12) Patent Application Publication
(10) Pub. No.: US 2026/0201360 A1
(43) Pub. Date: Jul. 16, 2026

(54) METHODS FOR NUCLEIC ACID EXTRACTION

(71) Applicant: STEMCELL TECHNOLOGIES CANADA INC., Vancouver (CA)

(72) Inventors: Jason DIXON, Vancouver (CA); Charlotte DEWAR, Vancouver (CA); Gary BRAUN, Vancouver (CA); Mark HILLS, Vancouver (CA); Ryan BAILEY, Vancouver (CA); Adil KASSAM, Vancouver (CA); Karina MCQUEEN, Vancouver (CA)

(21) Appl. No.: 19/135,501

(22) PCT Filed: Dec. 14, 2023
(86) PCT No.: PCT/CA2023/051662

§ 371 Date: Jun. 4, 2025

Related U.S. Application Data
(60) Provisional application No. 63/432,862, filed on Dec. 15, 2022.

(51) Int. Cl. C12N 15/10

(52) U.S. Cl. CPC C12N 15/1013

(57) ABSTRACT

The present disclosure relates to particle-based methods and kits for extracting nucleic acids, such as with magnetic or magnetically-responsive particles, from a wide variety of biological samples. The methods and kits of this disclosure may be used to extract nucleic acids with high purity and recovery (compared to conventional methods, such as using spin-columns) from simple through complex biological samples. The methods and kits of this disclosure may be used in high-throughput extractions or may be amenable to use in high-throughput applications.

### CROSS-REFERENCE TO RELATED APPLICATION

This application claims the benefit of U.S. Provisional Patent Application No. 63/432,862 filed Dec. 15, 2022, the entire content of which is hereby incorporated by reference in its entirety.

### TECHNICAL FIELD

This disclosure relates to nucleic acid extraction, and more specifically to methods for extracting nucleic acids from biological samples, and still more specifically to methods for extracting nucleic acids from a wide range of simple and complex biological samples.

### BACKGROUND

Methods and/or kits for isolating nucleic acids are commercially available. However, many such methods and kits fail to efficiently isolate nucleic acids from a wide range of biological samples, including simple and complex samples. Spin column-based approaches of nucleic acid extraction, though may be preferred by convention, tend to not scale out well and yield low nucleic acid purity and recovery. Particle-based approaches to extracting nucleic acids are not designed for wide ranges of biological samples and do not perform well when extracting nucleic acids from more complicated samples, such as whole blood, organoids and whole organs, typically suffering from low purities and recoveries.

In the diagnostic and medical field, reliable and efficient kits and method are needed to isolate nucleic acids of optimal purity and recovery from a broad range of biological samples, such as from simple and complex biological samples, and that are amenable to efficiently extracting nucleic acids from a large number of samples in a relatively short amount of time. Nucleic acid extraction methods suitable for automation would also offer great benefit in such settings.

Therefore, there is a particular need for improved kits and methods for extracting nucleic acids from a broad range of biological samples, such as simple samples like single cell suspensions and complex samples like whole blood or tissues.

### SUMMARY

The present disclosure relates to kits and/or methods for extracting nucleic acids from a biological sample.

In one aspect of this disclosure are provided methods for extracting nucleic acids from a biological sample. The methods of this disclosure may comprise i) preparing a sample lysate by treating the biological sample with a lysis buffer; ii) incubating the sample lysate at a temperature at or above room temperature and below 60° C.; iii) adding particles to the sample lysate to bind the nucleic acids; iv) separating the particles and the nucleic acids bound thereto from the sample lysate; and v) contacting the particles and the nucleic acids bound thereto with an elution buffer to release the nucleic acids from the particles.

In one embodiment, the particles are magnetic particles, and the magnetic particles are separated from the lysate in the presence of a magnetic field.

In one embodiment, a purity and/or recovery of the released nucleic acids are higher than a purity and/or recovery of the released nucleic acids when the biological sample of above step i) is incubated above room temperature compared to at room temperature.

In one embodiment, the pH of the lysis buffer is between 4 and 10. In one embodiment, a purity of eluted nucleic acids is not compromised when the pH of the lysis buffer ranges between 6 and 10.

In one embodiment, a method of this disclosure may further comprise pre-heating the elution buffer to a temperature above room temperature (RT) and below 60° C. In one embodiment, the temperature of the elution buffer is between 30° C. and 60° C. In one embodiment, pre-heating the elution buffer improves purity and/or recovery of the released nucleic acids compared to the purity and/or recovery of the nucleic acids released in room-temperature elution buffer.

In one embodiment, the particles are added to the sample lysate at a concentration above 1 mg/ml of the sample lysate and below 2.2 mg/ml of the sample lysate. In one embodiment, the concentration of the particles is between about 1.2 mg/ml of the sample lysate and 2 mg/ml of the sample lysate.

In one embodiment, the biological samples is whole blood, suspended cells, isolated cells, PBMCs, liver tissue, extracellular vesicle, leukapheresis product, single cell suspension, organoids, plasma, and virus.

In one embodiment, the biological sample is a complex sample.

In one embodiment, the biological sample is pre-processed. In one embodiment, the biological sample is not pre-processed.

In one embodiment, the biological sample comprises between 10¹ and 10⁹ cells.

In one embodiment, a method of this disclosure may further comprise subjecting the released nucleic acids directly to one or more downstream applications.

In one embodiment, a method of this disclosure may further comprise adding fresh particles to the released nucleic acids to recapture the released nucleic acids.

In one embodiment, a method of this disclosure may further comprise releasing the recaptured nucleic acids, using an elution buffer of this disclosure.

In one embodiment, the method does not involve washing the particles and the nucleic acids bound thereto before contacting them with the elution buffer.

In one embodiment, the method does not involve washing the first and/or second particles and the nucleic acids bound thereto.

In one embodiment, a purity and/or recovery of the recaptured nucleic acids released from the fresh particles is higher than a purity and/or recovery of nucleic acids released in step v) stated above.

In one embodiment, a method of this disclosure may further comprise fractionating the particles and a supernatant comprising the released nucleic acids by aspiration or pour-off.

In one embodiment, the particles are silica-based.

In one embodiment, the method elapses between 30-45 minutes.

In another aspect of this disclosure are provided kits for extracting nucleic acids from a biological sample. Kits of this disclosure may comprise a lysis buffer (as described herein), one or more vials of particle (as described herein), a wash buffer (as described herein), and an elution buffer (as described herein). In one embodiment kits of this disclosure further comprise a priming buffer (as described herein).

Other features and advantages of the presently described subject-matter will become apparent from the following detailed description. It should be understood, however, that the detailed description and the specific examples while indicating preferred embodiments of the described subject-matter are given by way of illustration only, since various changes and modifications within the spirit and scope of the invention will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows bar graphs for purity and recovery of nucleic acids extracted from leukopak PBMCs when the pH of the lysis buffer used in a method of this disclosure varied from 4 to 10. 260/280 and 260/230 absorbance ratio values obtained from a spectrophotometric instrument assessed purity (salt and protein contaminants) of extracted DNA (A). Amount (μg) of DNA and RNA extracted per 1×10⁶ leukopak PBMCs as measured using a Qubit Fluorometer (B). Error bars represent the Mean±S.D of 2 technical replicates.

FIG. 2 shows boxplots for purity and recovery of nucleic acids extracted from unwashed leukopak cells from three human donors when only the concentration of magnetic particles in 90% of isopropyl alcohol was varied in a method of this disclosure: 1, 1.2, 1.4, 1.6, 1.8, 2, 2.2 and 2.4 mg/ml of sample lysate. 260/230 absorbance ratio values assessed purity (salt and protein contaminants) of extracted nucleic acids (A). Amount (in ng/μL) of nucleic acids extracted per 1×10⁶ leukopak cells as measured using a Qubit Fluorometer (B).

FIG. 3 compares the recovery of RNA extracted from a sample of hepatic organoids using a conventional spin columns (“Spin”) or an exemplary particle-based workflow of this disclosure (“EasySep™”). Hepatic organoid lysates were prepared using either a phenol-chloroform-based reagent (TRIzol), or a lysis buffer of the present disclosure (IH-LB). Data points represent three technical replicates.

FIG. 4 shows UV spectrophotometric absorption spectra for nucleic acids extracted from a sample of whole blood using a standard workflow, spin column workflow and an improved workflow according to an exemplary embodiment of the present disclosure.

FIG. 5 compares the purity and recovery of nucleic acids extracted from a sample of whole blood using a two-step particle-based workflow of the present disclosure (“Improved Workflow”). Control nucleic acid extraction workflows included pre-lysed RBC samples and a spin column-based method. Conditions 1~4 comprised re-capturing eluted nucleic acids to the same particles as in the “Improved Workflow”, but varied the number of wash steps prior to elution and re-capture as follows: no wash (condition 1), 1×wash (condition 2), 2×wash (condition 3), and 3×wash (condition 4). Purity (260/230) of re-captured nucleic acids was determined by UV spectrophotometry (A), and recovered quantity (μg) of nucleic acids extracted per 1×10⁶ input cells was measured using a NanoDrop2000 (B). n=1 in duplicate.

FIG. 6 shows UV spectrophotometric absorption spectra for nucleic acids extracted from a sample of whole blood according to a two-step method of this disclosure, in either the presence or absence of priming buffer to prime a first elution of nucleic acids prior to re-capturing the nucleic acids by freshly added particles.

FIG. 7 shows bar graphs comparing the purity and recovery of nucleic acids extracted from a sample of whole blood using a two-step method of this disclosure (“Improved”), a standard one-step particle-based workflow (e.g. single particle addition) (“Standard”), and conventional spin-column extraction. 260/230 absorbance ratio values obtained from a spectrophotometric instrument assessed purity (salt and protein contaminants) of extracted nucleic acids (A). n=3. Error bars represent±1 standard deviation. Amount (μg) of DNA (B) and RNA (C) extracted per 1×10⁶ cells as measured using a Qubit Fluorometer. n=7. Error bars represent±1 standard deviation.

### DETAILED DESCRIPTION

The present disclosure relates to methods and/or kits for extracting nucleic acids from a biological sample. More specifically, this disclosure relates to extracting nucleic acids from a range of biological samples, such as samples that range from simple samples to more complex samples, by practicing the disclosed methods and/or by using the kits. Still more specifically, this disclosure relates to high-throughput extraction of nucleic acids from a range of biological samples, such as samples that range from simple samples to more complex samples.

Where used in this disclosure, the term “nucleic acid” refers to linear, branched or circular DNA and RNA. Examples of RNA may include mRNA, siRNA, miRNA, snRNA, tRNA, hnRNA, ribozymes, viral RNA, or extracellular or cell-free RNA. Examples of DNA may include plasmid DNA, genomic DNA, mitochondrial DNA, viral DNA, and extracellular or cell-free DNA.

Where used in this disclosure, the term “particles” refers to particles that reversibly bind nucleic acids, such as DNA and/or RNA. In a specific embodiment, the particles are magnetic particles or magnetically-responsive particles, and may more specifically be referred to as ferromagnetic, ferrimagnetic, paramagnetic or superparamagnetic particles. Magnetic or magnetically-responsive particles may comprise a magnetic rich core encapsulated by a polymer shell. Any particles of this disclosure, including magnetic or magnetically-responsive particles, are available commercially and would be well known to a person skilled in the art. Particles may may be silica coated particles, and the silica may be in the form of silica gel, silica particles, silica fibres, siliceous oxide, silicon dioxide, alkylsilica, aluminium silicate, borosilicate, solid silica such as glass or diatomaceous earth, glass fibres, or a mixture of two or more of the above. The silica should be present at least in part on the surface of the beads. For efficient binding, it is preferred that the silica is present about all or substantially most (e.g. >50%, >60%, >70%, >80%, >90%, or >95%) of the particle surface. In one embodiment, the siliceous-oxide coated (magnetic) particles also have an adsorptive surface of hydrous siliceous oxide. The target nucleic acid, such as DNA or RNA, adheres to the adsorptive surface of the particles while the binding of other material from a sample lysate, particularly deleterious contaminants such as nucleases, may be limited.

In one embodiment, particles (e.g. magnetic particles) of this disclosure function as a solid phase. The term solid phase encompasses appropriate materials that are porous or non-porous, or permeable or impermeable. In one embodiment, the surface of the solid phase (e.g. a silica solid phase) is not modified, such as with functional groups. In one embodiment, the surface of the solid phase (e.g. a silica solid phase) is modified, such as with functional groups. In one embodiment, the solid phase may carry anion exchange functional groups which can bind the nucleic acid of interest.

Where used in this disclosure, the term “chaotropic agent” refers to agents that disrupt the hydrogen bonding network in water solution, which may destabilize the native state of macromolecules (e.g. proteins, nucleic acids) in the solution. A chaotropic agent denatures nucleic acid-associated proteins, therefore weakening the hydrophobic interaction between such proteins and nucleic acids. Non-limiting examples of a chaotropic agent include guanidinium thiocyanate, guanidine, urea, and thiourea. Preferably, a chaotropic agent is a guanidinium salt (e.g. guanidinium hydrochloride, guanidinium thiocyanate, and/or guanidinium isothiocyanate). In one embodiment, a concentration of the chaotropic agent in a buffer, such as a lysis buffer, may range between ≥0.1 M to ≤10 M, ≥1 M to ≤8 M, ≥3 M to ≤7 M, or ≥4 M to ≤6 M.

Where used in this disclosure, the term “purity” or “purity ratio” refers to a purity of extracted nucleic acids, as may be assessed from 260/280 and 260/230 absorbance ratios. Pure DNA has an A260/A280 nm ratio with an acceptable range of 1.7-2.0, while pure RNA has an A260/280 ratio with an acceptable range of 1.9-2.2. If the 260/280 ratio is appreciably lower (≤1.6), it may indicate the presence of proteins, phenols, or other contaminants that absorb at or near 280 nm. Expected A260/A230 values are commonly in the range of 1.8-2.2 for each of DNA and RNA. If the 260/230 ratio is outside of the foregoing range, it may indicate the presence of contaminants (e.g., proteins, carbohydrates, lipids, salts, EDTA, or phenol) which absorb at 230 nm. The 260/230 ratio is widely used as a secondary measure of nucleic acid purity.

Where used in this disclosure, the term “recovery” refers to an amount or concentration (such as in ng or in μg) of extracted or isolated nucleic acids (e.g. DNA and/or RNA), and may be expressed relative to an input number of cells. Nucleic acid recovery is usually measured with a spectrophotometer, microspectrophotometer, or fluorometer.

### Methods

In one aspect of this disclosure are provided methods for extracting nucleic acids from a biological sample. In embodiments, methods of extracting nucleic acids from a biological sample may be performed on a broad range of biological samples, such as simple and complex samples. In embodiments, methods of extracting nucleic acids from a biological sample may be performed on a broad range of biological samples, such as simple and complex samples without substantially sacrificing nucleic acid purity and/or recovery.

Biological samples of this disclosure are not particularly limited, provided that they comprise nucleic acids. Thus, biological samples may be of prokaryotic, viral, or eukaryotic origin. In a specific embodiment, the biological sample is mammalian in origin. Biological samples may be fresh, frozen, desiccated, or cryopreserved. Methods (and kits) of this disclosure may be performed on a broad range of biological samples, ranging from relatively more simple samples to relatively more complex samples.

Where used in this disclosure, the term “simple biological sample” may refer to any sample that in terms of the presence/concentration of protein, carbohydrate, lipid, chemical or other contaminants is simple in its composition and/or requires minimal or no pre-processing or special treatment before extracting nucleic acids. Examples of simple biological samples include but are not limited to (enriched) cells suspended in a buffer or medium (e.g. single cell suspensions), plasma, leukapheresis product (e.g., PBMCs), and the like.

Where used in this disclosure, the term “complex biological sample” may refer to any sample that in terms of the presence/concentration of protein, carbohydrate, lipid, chemical or other contaminants is complex in its composition and/or requires pre-processing or special treatment before extracting nucleic acids. It can be seen that the concepts of simple biological samples and complex biological samples are relative terms, with the latter comprising more contaminants than the former, particularly those contaminants which the skilled person would understand could impact purity and/or recovery in particle-based nucleic acid extraction. A complex sample may comprise few or many contaminants, together at relatively high concentrations. Examples of contaminants in the simple and/or complex samples may include salts, proteins, cells, and endogenous and/or exogenous small organic molecules (e.g., lipids, amino acids). In particular, a source of contaminants may come from cellular materials that are not nucleic acids, such as proteins, lipids, carbohydrates, or other macromolecules, intact cells, or cell debris. Accordingly, a relatively high concentration of such contaminants may reduce the efficiency of nucleic acid extraction, particularly by reducing purities and recoveries. Examples of complex biological samples include but are not limited to blood (e.g., whole blood, a crude whole blood lysate, serum, plasma, or menstrual blood), bloody fluids (e.g., wound exudate, phlegm, bile, and the like), tissue samples (e.g., tissue biopsies, skin biopsies, muscle biopsies, or lymph node biopsies), whole organs (e.g., liver), homogenized tissue samples, urine, CSF, BAL, SF, semen, feces, sputum (e.g., purulent sputum or bloody sputum), organoids and the like.

A number of cells in the biological sample from which nucleic acids are to be extracted using methods (and kits) of this disclosure may vary. In one embodiment, a number of cells in the biological sample can be on the order of 10¹, 10², 10³, 10⁴, 10⁵, 10⁶, 10⁷, 10⁸, or 10⁹.

In one embodiment, the biological sample is whole blood, which may be considered a complex sample because it comprises RBCs, WBCs, platelets, plasma, and the respective cytosol, macromolecules, and small molecules thereof. In one embodiment, whole blood comprises anti-coagulants.

In one embodiment, the biological sample is a sample of PBMCs or a leukapheresis product. PBMC samples may comprise relatively low levels of or no contaminating RBCs, granulocytes, and/or platelets. In one embodiment, the biological sample is plasma.

In one embodiment, the biological sample comprises viruses and/or prokaryotes. In one embodiment, the biological sample comprises soil and/or plant matter.

In one embodiment, the biological sample is a suspension of cells in a suitable buffer or medium. The suspension of cells may be isolated or enriched using an isolation or enrichment method, such as by immunomagnetic separation, centrifugation (e.g. density gradient centrifugation), or filtration.

In one embodiment, the biological sample is a leukapheresis product. In one embodiment, the leukapheresis product is adjusted with a suitable buffer to obtain a desired cell concentration for nucleic acid extraction.

In one embodiment, the biological sample is an organoid or comprises organoids. Organoids may be comprised in or on an extracellular matrix (e.g. Matrigel dome), thus an organoid sample may comprise one or more extracellular matrix proteins. If the organoids are comprised in or on an extracellular matrix, they may be broken down in complexity by incubation in a lysis buffer (e.g. a phenol-chloroform solution, or an enzymatic solution) and/or mechanically triturated using a pipette.

In one embodiment, the biological sample is a whole organ or a fragment of an organ. The organ or fragment may be broken down in complexity (e.g. dissociated or homogenized) via one or more of mincing, mechanical trituration, incubation in an enzymatic solution, or otherwise.

Thus, the present disclosure relates to methods of extracting nucleic acids from any available biological sample, provided the sample comprises nucleic acids to be extracted and/or analyzed.

Methods of this disclosure may comprise preparing a sample lysate by treating the biological sample with a lysis buffer. Many lysis buffers are known in the art. A sample lysate may be prepared in a lysis buffer comprising one or more of:
a) Proteinase K, such as at a concentration between 10-100 μl/ml of sample, to cleave peptide bonds and digest proteins.
b) One or more detergents (e.g SDS, Tween-20 and Sarkosyl), such as at a concentration between 3% to 20%, to lyse cells (release soluble proteins) by solubilizing membrane proteins and lipids.
c) High osmolarity or osmolality, to impart osmotic pressure on cells and cause water to enter the cell, thereby increasing its internal pressure and resulting in cell lysis.
d) One or more buffering agent to maintain a desired pH (e.g. 6.0-10.0), such as Tris, HEPES, sodium bicarbonate, ACES, PIPES, MPSO, imidazole, MOPS, triethanolamine, pyrophosphate, sodium chloride, deoxycholate, or any combination thereof.
e) A binding inducing agent, such as a chaotropic agent or a chaotropic salt, which may be provided alone or in combination with a binding additive, such as an alcohol (e.g. ethanol or isopropanol).

In one embodiment, a sample lysate is prepared in a lysis buffer not comprising Proteinase K.

pH of the lysis buffer may range between 3 and 12, or between about 4 and 10. In one embodiment, pH of the lysis buffer is about 6, about 7, about 8, about 9, or about 10. In one embodiment, a purity of released nucleic acids is not compromised when the pH of the lysis buffer falls in the range between 3 and 12, or more preferably between about 4 and 10.

A sample lysate will comprise nucleic acids among other cellular components. Examples of other cellular components may include fragments of cell and/or nuclear membranes, organelles, and macromolecules such as carbohydrates, proteins, and lipids. The nature of components in the milieu of the sample lysate may determine if the biological sample is a complex biological sample or a simple biological sample, as defined above.

The sample lysate may be prepared by heating the treated sample (or the lysis buffer) to a temperature at or above room temperature and below 60° C. In one embodiment, the sample lysate is prepared by heating the treated sample (or the lysis buffer) to a temperature above 30° C. and below 60° C. In one embodiment, the sample lysate is prepared by heating the treated sample (or the lysis buffer) to a temperature above 37° C. and below 60° C. In one embodiment, the sample lysate is prepared by heating the treated sample (or the lysis buffer) to a temperature that is preferably about 56° C.±3° C. In one embodiment, the sample lysate is prepared by heating the treated sample (or the lysis buffer) to a temperature that is about room temperature±3° C.

The sample lysate may be prepared by incubating at a temperature range described in the foregoing, or in a lysis buffer heated to a temperature range described in the foregoing. The sample lysate may be incubated for any amount of time provided that the nucleic acids do not begin to significantly degenerate. In one embodiment, the sample lysate is prepared by incubating at a given temperature (as described above) for between about 3-30 minutes, between about 5-20 minutes, or between about 10-15 minutes. In one embodiment, the sample lysate is prepared by incubating at a given temperature (as described above) for about 5 minutes, about 10 minutes, or about 15 minutes.

Methods of this disclosure comprise adding particles, such as magnetic particles, to the sample lysate to bind the nucleic acids. It may be necessary to incubate the sample lysate in the presence of the particles, to allow the nucleic acids to become complexed or bound thereto. In one embodiment, a volume of the particles is added at about the same proportion to the sample volume (e.g. at a 1:1 ratio). In one embodiment, a volume of the particles is added at a different proportion to the sample volume (e.g. 0.5:1 or 1.5:1, or 1:0.5 or 1:1.5). In one embodiment, incubation is performed at room temperature. In one embodiment, incubation is performed for about 5 minutes. In one embodiment, incubation is for less than 5 minutes. In one embodiment, incubation is for about 3 minutes. In one embodiment, incubation is for about 1 minute.

The particles may in addition to binding nucleic acids also non-specifically bind cellular components other than nucleic acids. In one embodiment, the particles may non-specifically bind proteins or other macromolecules in a sample lysate (e.g. a cell lysate). In one embodiment, the particles may non-specifically bind hemoglobin in the sample/cell lysate. The nature of the contaminants in the sample lysate, and thus the substances that may non-specifically be bound to the particles, may determine whether the biological sample is a complex biological sample or a simple biological sample.

Methods of this disclosure may comprise separating the particles and the nucleic acids bound thereto to enrich the nucleic acids in the sample lysate from proteins and other debris in the sample lysate. Separation may occur in any way that enables the separation/isolation of the particles. By way of non-limiting example, the particles may be centrifuged to form a pellet, or if the particles are magnetic particles they may be exposed to a magnetic field for collection against a container surface closest to the magnetic field.

In a specific embodiment where the particles are magnetic particles, the particles and the nucleic acids bound thereto are separated from a sample lysate by applying a magnetic field to the sample lysate. It may be necessary to incubate the particles for a minute or longer in the presence of the magnetic field, to maximize the co-localization of the particles such as against a wall of the container/tube most proximal to the magnet/magnetic field. In one embodiment, incubation is performed at room temperature. In one embodiment, incubation is performed for about 5 minutes. In one embodiment, incubation is for less than 5 minutes. In one embodiment, incubation is for about 3 minutes. In one embodiment, incubation is for about 1 minute.

Following collection of the particles (e.g. magnetic particles) on or along a surface of a container (closest to a magnetic field), a supernatant may be removed. It may be important that the container is maintained in proximity of the magnetic field, so that the particles may be fractionated from the balance of the sample lysate. In one embodiment, the supernatant may be removed by a pipette. In one embodiment, the supernatant may be poured off.

Once the particles have been fractionated from the supernatant, it may be important to wash the particles to remove contaminating materials entrained among the particles and/or contaminating materials weakly interacting with or bound to the particles. In one embodiment, the pellet is washed with a wash buffer such as buffer RW1 (Qiagen), AW1 (Qiagen), PBS, EasySep™ Buffer (STEMCELL Technologies), or any traditional wash buffer containing a high concentration of ethanol (or isopropanol). In one embodiment, the wash buffer comprises an organic solvent such as lower alcohols (i.e., C1-5 alcohols). In one embodiment, the wash buffer comprises ethanol. In one embodiment, the wash buffer comprises 40-80% ethanol or isopropanol.

Any number of wash cycles may be performed to increase purity; however, increasing the number of washes may come at the expense of recovery. In one embodiment, the pellet is washed with a wash buffer once. In one embodiment, the pellet is washed with a wash buffer twice. In one embodiment, the pellet is washed with a wash buffer three times. In one embodiment, the pellet is washed with a wash buffer more than three times, such as five times or more.

The pH and solute composition and concentration of the wash buffer solution can be varied according to the type of impurities which are expected to be present.

Methods of this disclosure may comprise contacting the particles and the nucleic acids bound thereto with an elution buffer to release the nucleic acids from the particles. Elution buffers are known, and when eluting from a silica matrix, as may be comprised on particles of this disclosure, elution efficiency may be influenced by pH. An elution buffer of this disclosure may comprise one or more of the following: a low-salt concentration; TE buffer or about 10-100 mM Tris-HCl; pH of about 8.0 or 8.5; and about 0.1-1 mM EDTA. Efficient elution of nucleic acids from the particles may be achieved by incubating the particles in water or a buffer with low ionic strength. In one embodiment, the nucleic acids bound to the particles are eluted using distilled or deionized or nuclease-free water.

Methods of this disclosure may further comprise pre-heating the elution buffer before contacting the particles and the nucleic acids bound thereto with the elution buffer. In one embodiment, the elution buffer is pre-heated to a temperature at or above room temperature and below 60° C. In one embodiment, the elution buffer is pre-heated to a temperature above 30° C. and below 60° C., or between 35° C. and 55° C., or between 40° C. and 50° C. In one embodiment, the elution buffer is pre-heated to 37° C.±3° C. In one embodiment, the elution buffer is pre-heated to 56° C.±3° C. In one embodiment, a method of this disclosure further comprises incubating the particles and the nucleic acids bound thereto at any of the foregoing temperatures after they are contacted with an elution buffer.

Pre-heating an elution buffer (or incubating particles in elution buffer at the foregoing temperatures) as described above may improve purity and/or recovery of released nucleic acids. In one embodiment, pre-heating the elution buffer improves purity of the released nucleic acids compared to the purity of nucleic acids released in room-temperature elution buffer. In one embodiment, pre-heating the elution buffer improves recovery of the released nucleic acids compared to the recovery of nucleic acids released in room-temperature elution buffer.

In one embodiment, the methods may comprise releasing preferentially the nucleic acids from the particles. Preferential elution may refer to those conditions under which nucleic acids are substantially or completely released from the particles, while other cellular components may remain bound. In one embodiment, about 90-100% of the nucleic acids are released from the particles. In one embodiment, about 80-90% of the nucleic acids are released from the particles. In one embodiment, about 70-80% of the nucleic acids are released from the particles. In one embodiment, about 60-70% of the nucleic acids released from the particles. In the same or different embodiments, about 90-100% of the other components remain bound to the particles. In one embodiment, about 80-90% of the other components remain bound to the particles. In one embodiment, about 70-80% of the other components remain bound to the particles. In one embodiment, about 60-70% of the other components remain bound to the particles. In one embodiment, about 50-60% of the other components remain bound to the particles. In one embodiment, about 40-50% of the other components remain bound to the particles.

In one embodiment, a method of this disclosure does not involve washing prior to the elution step. In one embodiment, the method does not involve washing the particles and the nucleic acids bound thereto. In one embodiment, the method involves minimal washing of the particles and the nucleic acids bound thereto, such as only a single wash.

Methods of this disclosure may further comprise fractionating the particles and a supernatant comprising the released nucleic acids. The particles and the released nucleic acids (in a supernatant) may be fractionated by aspiration or pour-off. The particles and supernatant may be fractionated in any way that co-localizes the particles, such as by pelleting the particles by centrifugation, or maintaining the particles if magnetic in the presence of a magnetic field during fractionation. In one embodiment, fractionation occurs in the presence of a magnet or magnetic field, and the supernatant (e.g. fraction comprising the nucleic acids) is transferred to a separate container or tube. Thus, the supernatant or the eluent may be referred to as the first elution.

Method of this disclosure may further comprise adding fresh particles to the released nucleic acids of a first elution to rebind/recapture the nucleic acids in the first elution. In one embodiment, methods of this disclosure comprise adding fresh particles to the released and fractionated nucleic acids of a first elution. Freshly added particles may be referred to as the second particles (e.g., second magnetic particles). Nucleic acids in the first elution may be incubated in the presence of second particles as described above, in terms of, for example, particle and first elution relative volumes, incubation time, incubation temperature, etc. In one embodiment, the volume of fresh particles added to the first elution comprises an alcohol, such as at a 40% concentration, 50% concentration, 60% concentration, 70% concentration, or higher.

Prior to rebinding/recapture of nucleic acids in a first elution to fresh/second particles, such as in a two-step extraction of this disclosure, the nucleic acids may be primed for recapture. A priming buffer may modify the conditions of the first elution to facilitate recapture of the nucleic acids, such as by restoring salt concentration, pH, conformation, etc.

Nucleic acids in the first elution may be contacted with a priming buffer, such as a buffer comprising one or more of: a chaotropic agent or chaotropic salt; an alcohol (e.g. at a high concentration); an agent of cell lysis (as described above); and a binding additive, such as an alcohol. In one embodiment, priming comprises topping up the first elution with the priming buffer, such as at a 1:1 ratio or a 1:1.5 ratio. In one embodiment, the alcohol is isopropanol. In one embodiment, the isopropanol used is 100%. In one embodiment, the priming buffer is same as the lysis buffer described herein.

In one embodiment, the nucleic acids in the first elution are primed (in a priming buffer) during contact with the second particles. First elution thus treated or primed prior to rebinding (eluted) nucleic acids to the second particles may be referred to as treated or primed samples.

Methods of this disclosure may further comprise separating the second particles and the nucleic acids bound thereto, essentially as described above. A second separation may further enrich the nucleic acids in the first elution from proteins, debris, or other residue in the first elution, such as may carry over to a first elution from a complex biological sample, or to concentrate the nucleic acids regardless of whether the biological sample is simple or complex.

After the second particles and the nucleic acids of the first elution have been separated from a balance of the first elution, washes may be performed, essentially as described above. In one embodiment, methods of this disclosure do not involve washing prior to a second elution step.

As described above with respect to the first elution, methods of this disclosure may further comprise contacting the second particles and nucleic acids bound thereto with an elution buffer. The nature of the elution buffer (e.g. components, temperature, etc), and the duration of the contacting step are essentially as described above.

Following release of recaptured nucleic acids from fresh/second particles, methods of this disclosure may further comprise fractionating the second particles and a supernatant comprising the nucleic acids, essentially as described above.

Particles of this disclosure are not particularly limited, as many vendors have commercialized particles for nucleic acid extraction. Nucleic acids may bind to first (magnetic) particles and/or second (magnetic) particles through various binding mechanisms. For example, the binding mechanism between nucleic acids and (magnetic) particles may be through electrostatic/ionic interactions. Or, In one embodiment, the binding mechanism between nucleic acids and (magnetic) particles may be through hydrophobic interactions. Or, the binding mechanism between nucleic acids and (magnetic) particles may be through hydrogen/dipole bonds interactions.

Particles (e.g., first and/or second particles), whether magnetic or otherwise, may be added to a sample lysate at any desired concentration. A concentration of the first particles and/or the second particles may be determined based on the volume of a sample lysate. In one embodiment, a concentration of the first particles and/or the second particles ranges between 0.1 μg/ml to 1 μg/ml of sample lysate. In one embodiment, a concentration of the first particles and/or the second particles ranges between about 1 g/ml to 10 μg/ml of sample lysate, about 1 μg/ml to 5 μg/ml of sample lysate, or about 1 μg/ml to 3 μg/ml of sample lysate. In one embodiment, the first particles and/or the second particles are Concentrated EasySep™ Total Nucleic Acid Rapidspheres™ (STEMCELL Technologies), and if concentrated the particles may need to be diluted in a sample lysate or prior to adding them to a sample lysate.

In one embodiment, the particles are added to a sample lysate at a concentration above 1 mg/ml of the sample lysate and below 2.2 mg/ml of the sample lysate. In one embodiment, a concentration of particles is between about 1.2 mg/mL of a sample lysate and 2 mg/ml of a sample lysate. In one embodiment, the particles are added at a concentration ranging between 0.5 and 3 mg, or between 0.75 and 2.75 mg, or between 1 and 2.5 mg, per mL of the sample lysate. In embodiments, a concentration of the particles added to a sample lysate is about 1.2 mg/ml of the sample lysate, or about 1.4 mg/ml of the sample lysate, or about 1.6 mg/ml of the sample lysate, or about 1.8 mg/ml of the sample lysate, or about 2.0 mg/ml of the sample lysate, or about 2.2 mg per ml of the sample lysate.

In embodiments, the nucleic acids of a first elution and/or a second elution may undergo further treatment, such as with a RNase solution (to yield purified or substantially purified DNA) or with a DNase (to yield purified or substantially purified RNA). In embodiments, a first elution and/or a second elution may be divided into separate containers, each container being respectively treated with a DNAse and an RNAse. In one embodiment, nucleic acids in a first elution and/or a second elution are not further treated.

The methods disclosed herein, whether a one-step particle addition method or a two-step particle addition method, yield nucleic acid purities that are high (e.g. within optimal A260/280 and/or A230/260 ranges). In one embodiment, purity of the nucleic acids released from second particles is higher than purity of the nucleic acids released from first particles. In one embodiment, purity of the nucleic acids released from second particles and purity of the nucleic acids released from first particles are both within optimal A260/280 and/or A230/260 ranges. In one embodiment, purity of the nucleic acids released from second particles is comparable to purity of the nucleic acids released from first particles (e.g. a purity within about ±1%, ±2%, ±3%, ±4%, ±5%, ±6%, ±7%, ±8%, ±9%, ±10%, ±12.5%, or ±15% of one another).

The methods disclosed herein, whether a one-step particle addition method or a two-step particle addition method, may yield nucleic acid purity higher than a purity of nucleic acids eluted from a conventional spin-column. In one embodiment, purity of the nucleic acids released from first and/or second particles and purity of the nucleic acids eluted from a conventional spin column are both within optimal A260/280 and/or A230/260 ranges. In one embodiment, purity of the nucleic acids released from first and/or second particles is within optimal A260/280 and/or A230/260 ranges, and purity of the nucleic acids eluted from a conventional spin column is not within optimal A260/280 and/or A230/260 ranges. In one embodiment, purity of the nucleic acids released from first and/or second particles is comparable to purity of the nucleic acids eluted off a spin column (e.g. a purity within about ±1%, ±2%, ±3%, ±4%, ±5%, ±6%, ±7%, ±8%, ±9%, ±10%, ±12.5%, or ±15% of one another).

Two-step methods of this disclosure may yield nucleic acid purity higher than a purity of nucleic acids released from a standard (one-step) particle-based workflow, whether commercially available or as disclosed herein. In one embodiment, purity of the nucleic acids released from second particles and the purity of the nucleic acids released from first particles are both within optimal A260/280 and/or A230/260 ranges. In one embodiment, purity of the nucleic acids released from second particles is within optimal A260/280 and/or A230/260 ranges, and purity of the nucleic acids released from first particles are not within optimal A260/280 and/or A230/260 ranges. In one embodiment, purity of the nucleic acids released from second particles is comparable to purity of the nucleic acids released from first particles (e.g. a purity within about ±1%, ±2%, ±3%, ±4%, ±5%, ±6%, ±7%, ±8%, ±9%, ±10%, +12.5%, or ±15% of one another).

Improvements in nucleic acid purity may be applicable to extractions from a wide range of biological samples, from simple to complex samples. In one embodiment, improvements in nucleic acid purity using a nucleic acid extraction method as disclosed herein are particularly applicable to extractions from complex biological samples, such as those samples that contain high levels of contaminants, without the need for pre-processing to reduce sample complexity.

The methods disclosed herein, whether a one-step particle addition method or a two-step particle addition method, yield high nucleic acid recovery (e.g. within 98%, or 95%, or 90%, or 85%, or 80%, or 75%, or 70%, or 65%, or 60%, or 55%, or 50% of the theoretical yield). In one embodiment, recovery of the nucleic acids released from second particles is higher than recovery of the nucleic acids released from first particles (e.g. about 5%, or 10%, or 15%, or 20%, or 25%, or 30%, or 40%, or 50%, or 60%, or 70%, or 80%, or 90%, or 2-fold higher). In one embodiment, recovery of the nucleic acids released from second particles is comparable to recovery of the nucleic acids released from first particles (e.g. within about ±5%, ±10%, +15%, ±20%, ±25%, ±30%, ±40%, or ±50% of one another).

The methods disclosed herein, whether a one-step particle addition method or a two-step particle addition method, may yield higher nucleic acid recovery than is eluted from a conventional spin-column (e.g. about 5%, or 10%, or 15%, or 20%, or 25%, or 30%, or 40%, or 50%, or 60%, or 70%, or 80%, or 90%, or 2-fold, or 3-fold, or 4-fold, or 5-fold higher). In one embodiment, recovery of the nucleic acids released from first and/or second particles is comparable to recovery of the nucleic acids eluted from a spin column (e.g. within about +1%, ±3%, ±5%, ±10%, ±15%, ±20%, ±25%, ±30%, ±35%, ±40%, ±45%, or ±50% of one another).

Two-step methods of this disclosure may yield nucleic acid recovery higher than a recovery of nucleic acids released from a standard (one-step) particle-based workflow, whether commercially available or as disclosed herein (e.g. about 5%, or 10%, or 15%, or 20%, or 25%, or 30%, or 40%, or 50%, or 60%, or 70%, or 80%, or 90%, or 2-fold higher). In one embodiment, recovery of the nucleic acids released from second particles is comparable to recovery of the nucleic acids released from first particles (e.g. within about ±5%, ±10%, +15%, ±20%, ±25%, ±30%, ±35%, ±40%, ±45%, or ±50% of one another).

Improvements in nucleic acid recovery may be applicable to extractions from a wide range of biological samples, from simple to complex samples. In one embodiment, improvements in nucleic acid recovery using a nucleic acid extraction method as disclosed herein are particularly applicable to extractions from complex biological samples, such as those samples that contain high levels of contaminants, without the need for pre-processing to reduce sample complexity.

In one embodiment, the nucleic acids extracted by the methods of this disclosure are directly used for downstream applications. In one embodiment, the extracted nucleic acids are directly used for downstream applications without a need for nucleic acid quantification. In one embodiment, the nucleic acids extracted are directly used for RT-qPCR without a need for DNAse treatment.

In one embodiment, a method of this disclosure does not involve the pre-processing of a biological sample, such as to simplify or clarify the biological sample, such as by filtration or the like. In the example of whole blood, the sample is not pre-processed to reduce or remove red blood cells and/or other contaminants comprised therein. In the example of a mucus comprising sample, it is not treated enzymatically, ultrasonically, or the like to liquefy the sample. Thus, in one embodiment, the only preparation of a biological sample may be to contact the sample with a lysis buffer.

Methods of this disclosure for extracting nucleic acids, whether in a one-step or two-step particle addition approach, may be high-throughput or amenable to high-throughput applications. Such methods may elapse between 20 and 45 minutes. In embodiments of extracting nucleic acids from a complex biological sample, and using a two-step particle addition approach as described above, methods of this disclosure elapse about 45 minutes or less, about 40 minutes or less, about 35 minutes or less, or about 30 minutes or less.

Methods of this disclosure may be carried out in any suitable container, including a tube (e.g. a microcentrifuge tube) or a plate (e.g. a microplate). Depending on the container used, a suitable magnet is used in embodiments where the particles are magnetic or responsive to a magnetic field. In one embodiment, a magnet is an EasySep™ magnet that receives tubes therein. In one embodiment, a magnet is a plate magnet that receives microplates thereon.

In one embodiment, methods of this disclosure offer various features and advantages compared to conventional nucleic acid extraction methods. In one embodiment, the method requires no pre-processing of the biological samples. In one embodiment, the first or second elution of nucleic acids may be used directly in downstream assays. In one embodiment, methods of this disclosure (whether one-step or two-step) extract nucleic acids of optimal purity and recovery, even if the starting biological sample is a complex samples.

The following non-limiting examples are illustrative of the present disclosure.

### EXAMPLES

### Example 1: Sample Preparation

Sample preparation methods were varied depending on the sample type.

Samples from a leukapheresis product were prepared by adjusting to a desired cell concentration. For example, a leukapheresis product can be diluted in D-PBS to 5×10⁶ cells/ml.

Samples of non-adherent cells were prepared by pelleting and resuspending in D-PBS to a desired concentration. Samples from 2D adherent cells were prepared in much the same way, except they were dissociated from the surface with a chosen dissociation reagent which may subsequently need to be quenched. Samples of cells isolated using an EasySep™ (STEMCELL Technologies) kit may be resuspended in an appropriate volume of D-PBS or EasySep™ buffer.

Whole blood samples were used directly for nucleic acid extraction. If not extracting nucleic acids immediately from a blood sample, it may be necessary to treat collected whole blood with an anticoagulant, such as ACD-A, heparin, or K-EDTA.

Organoids maintained in dome cultures (at least 4-6 confluent domes) were used for nucleic acid extraction. Briefly, organoids in Matrigel domes were mechanically triturated with an electronic pipette to break up the dome and fragment the organoids. Resulting cells were pelleted and resuspended in RLT buffer (Qiagen), a phenol chloroform-based solution (e.g. TRIzol), or a lysis buffer of this disclosure. Alternatively, a proteinase K containing lysis buffer was added directly to the dome, incubated and pipetted up and down to dissolve the dome.

Samples of liver tissue (e.g., mouse) were prepared by mincing harvested mouse liver into small pieces in a dish containing cold DMEM/F-12. After liver pieces settle down by gravity on ice for 2 minutes, supernatant was removed. 10 ml of RT Tissue Dissociation Cocktail (STEMCELL Technologies) was added to the liver pieces and incubated at 37 C water bath for 20 minutes.

Samples of PBMC were prepared from unprocessed human whole blood. To avoid loss of monocytes, EDTA was added to the whole blood sample to a final concentration of 6 mM prior to labelling and separation. Samples of PBMC were prepared directly from human cord blood and leukapheresis samples by immunomagnetic negative selection (STEMCELL Technologies).

Plasma samples were prepared by centrifuging whole blood at 2000×g for 10 minutes and transferring the plasma layer to a tube, which was centrifuged for an additional 10 minutes at 2000×g. The plasma supernatant was transferred to a new tube and centrifuged at 10,000×g for 30 minutes to remove cellular debris and large vesicles. The resulting supernatant was transferred to a required tube, and was used to isolate nucleic acids. For nucleic acid extractions from pan-extracellular vesicles (EVs), EVs were isolated from plasma using an appropriate EasySep™ kit (STEMCELL Technologies).

Samples prepared as above were used at a quantity/volume that depended on whether a microcentrifuge tube or 96-well plate was used. For extraction using a microcentrifuge tube, 25-300 μl of samples was used. For extraction using 96-well plate, 10-50 μl of samples was used.

### Example 2: Quantification and Analysis of Nucleic Acids

Nucleic acids extracted according to a method of this disclosure (see Example 3) and by other conventional methods were tested for purity and recovery. Average purity ratios, 260/280 and 260/230 were measured using a NanoDrop2000 (ThermoFisher). Recovery (μg) of nucleic acids (DNA and RNA) was measured using a Qubit™ Fluorometer (ThermoFisher).

### Example 3: Total Nucleic Acid Extraction

Samples prepared essentially as described in Example 1 were subjected to the following nucleic acid extraction method. Summarizing briefly the detailed description above, the samples were treated with 1:1 proportion of a proteinase K-containing lysis buffer, regardless of whether a microcentrifuge tube or 96-well plate was used. The sample was incubated for approximately 10 minutes, either at room temperature or in a 56° C. heat block or water bath, and the sample lysate was contacted with a volume of first magnetic particles (e.g. Diluted EasySep™ Nucleic Acid Rapidspheres™, STEMCELL Technologies), optionally in an alcohol, at a 1.5:1 ratio to the sample volume. The sample lysate and particles were incubated at room temperature for about 2-5 minutes to enable binding of the nucleic acids (e.g. a first capture). The first capture was enriched under the influence of a magnetic field for about 2-5 minutes at room temperature. After the supernatant was removed, whether by pipette or pour-off, the enriched first capture was resuspended in a suitable elution buffer, such as a TE buffer or nuclease-free water, for about 5 minutes at room temperature. The eluted nucleic acids were fractionated from the particles by either centrifugation or exposing the liberated particles to a magnetic field.

In an improved workflow, such as for complex samples, the nucleic acids in the eluant were primed or treated by exposure to a priming buffer comprising a chaotropic agent or chaotropic salt and optionally an alcohol, and contacted with a volume of second magnetic particles (e.g. Diluted EasySep™ Nucleic Acid Rapidspheres™), optionally in an alcohol, at a 1.5:1 ratio to the elution volume, for about 2-5 minutes at room temperature (e.g. a second capture). The second capture was enriched under the influence of a magnetic field for about 2-5 minutes at room temperature. The second capture was washed with a suitable wash buffer, such as PBS or the like, under a magnetic field about 1-3 times.

Eluted first and second captures can be quantified/analyzed as described in Example 2.

### Example 4: Effect of Proteinase K Lysis Temperature on the Purity and Recovery of Nucleic Acids

Leukapharesis samples prepared as described in Example 1 were treated with a lysis buffer containing Proteinase K at different temperatures (RT or 56° C.) to investigate the effect on purity and recovery of nucleic acids, in accordance with Example 3.

Samples treated with Proteinase K and incubated at 56° C. yielded better 260/280 and 260/230 ratios relative to samples incubated at RT. Recovery of nucleic acids (total nucleic acids and DNA) also improved among samples incubated at 56° C. relative to RT (Tables 1A and 1B)

TABLE 1A
Proteinase K lysis of leukopak samples at 56° C. for 10 minutes.

| | | Nanodrop | | | QuantIT | | |
|---|---|---|---|---|---|---|---|
| Wash | Wash Buffer | | | Conc | Total NA | DNA | RNA |
| Buffer 1 | 2 + 3 | 260/280 | 260/230 | (ng/uL) | (ng/ul) | (ng/uL) | (ng/uL) |
| Diluted LB + | 80% EtOH | 1.9 | 1.8 | 70 | 74 | 56 | 18 |
| 40% IPA | | | | | | | |
| IPA | 80% EtOH | 1.8 | 1.2 | 46 | 45 | 31 | 14 |
| 80% EtOH | 80% EtOH | 1.8 | 1.4 | 49 | 59 | 47 | 12 |
| 70% EtOH | 70% EtOH | 1.8 | 1.6 | 69 | 52 | 41 | 11 |

TABLE 1B
Proteinase K lysis of leukopak samples at RT for 3 minutes.

| | | Nanodrop | | | QuantIT | | |
|---|---|---|---|---|---|---|---|
| Wash | Wash Buffer | | | Conc | Total NA | DNA | RNA |
| Buffer 1 | 2 + 3 | 260/280 | 260/230 | (ng/uL) | (ng/ul) | (ng/uL) | (ng/uL) |
| Diluted LB + | 80% EtOH | 1.6 | 0.9 | 31 | 30 | 19 | 11 |
| 40% IPA | | | | | | | |
| IPA | 80% EtOH | 1.5 | 0.7 | 73 | 37 | 26 | 11 |
| 80% EtOH | 80% EtOH | 1.4 | 0.5 | 43 | 49 | 33 | 16 |
| 70% EtOH | 70% EtOH | 1.6 | 0.7 | 52 | 44 | 33 | 11 |

### Example 5: Effect of Lysis Buffer pH on the Purity and Recovery of Nucleic Acids

PBMC samples were prepared as described in Example 1 and treated with a lysis buffer having different pH levels to investigate the effect on purity and recovery of nucleic acids, in accordance with Example 3.

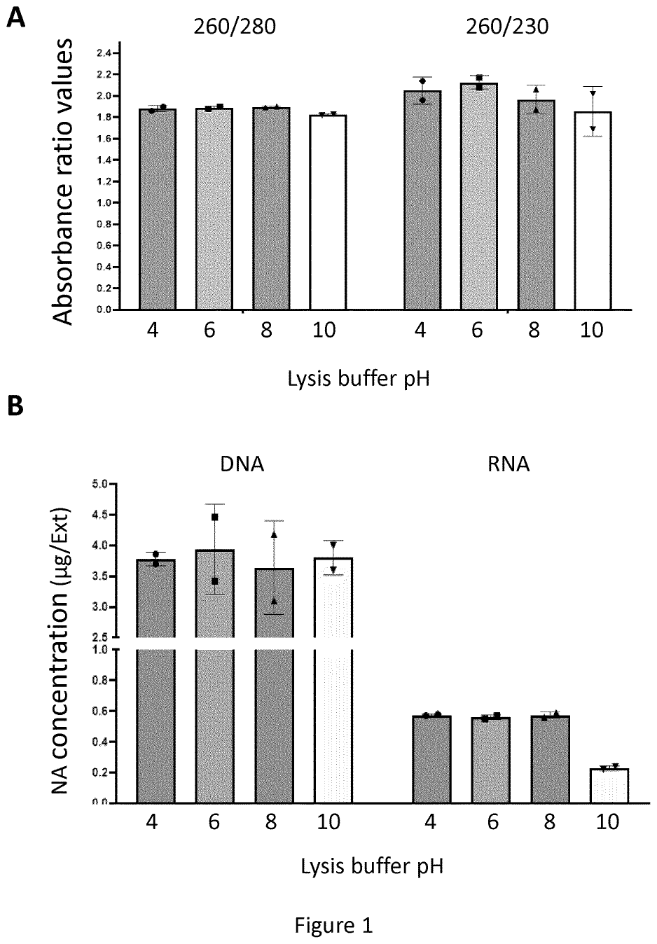

Comparable 260/280 and 260/230 ratios of extracted nucleic acids were obtained regardless of lysis buffer pH (e.g. 4.0, 6.0, 8.0 and 10.0) (FIG. 1A). DNA and RNA recovery from a sample lysed at pH 4.0, 6.0, 8.0 and 10.0 was comparable (FIG. 1B).

Thus, a lysis buffer of this disclosure at a pH from 4.0 and 10.0 did not compromise the purity and recovery of extracted nucleic acids.

### Example 6: Effect of Particle Concentration on the Purity and Recovery of Nucleic Acids

Unwashed cells of leukapharesis samples from 3 donors were prepared as described in Examples 1 and 3, and contacted with different concentrations of particle (1, 1.2, 1.4, 1.6, 1.8, 2.0, 2.2 and 2.4 mg of particles (in 90% IPA) per ml of sample lysate) ahead of nucleic acid extraction as described in Example 3

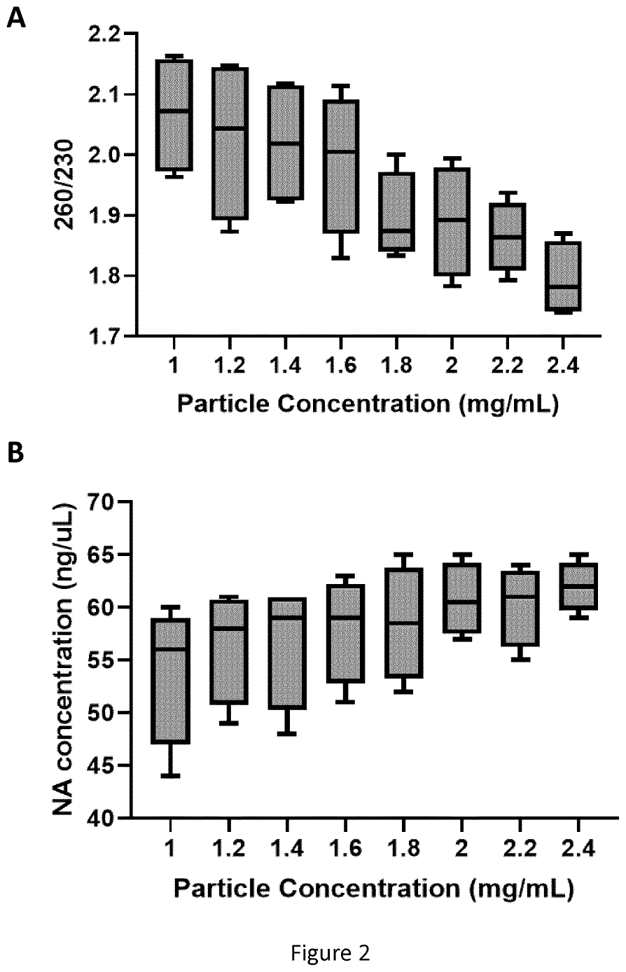

While recovery generally correlated with particle concentration, purity inversely correlated with particle concentration. While purity was best at particle concentrations of 1.6 mg/ml of sample lysate or higher, concentrations of 2.2 mg/ml of sample lysate and lower yielded purity within the acceptable range (FIG. 2A). Further, particle concentrations of 1.2 mg/ml of sample lysate and higher were more or less comparable (FIG. 2B).

Overall, particle concentration ranges between and including 1 to 2.2 mg/ml of sample lysate strike a balance between maximizing purity and recovery, and more specifically between 1.2 and 2 mg/ml of sample lysate.

### Example 7: Effect of Elution Temperature on the Purity and Recovery of Nucleic Acids

Whole blood samples were prepared as described in Example 1 and bound to particles as described in Example 3. The effect of elution buffer temperature (e.g. RT, 37° C., or 56° C.) on nucleic acid purity and recovery was investigated, relative to RT elution in a spin-column protocol.

While recovery generally correlated with elution buffer temperature, and exceeded spin column recovery at 56° C., 260/280 purity inversely correlated with elution buffer temperature.

TABLE 2
Effect of elution temperature on purity
and recovery of DNA from whole blood.

Whole blood

| | AL - RT | RT | 37° C. | 56° C. |
|---|---|---|---|---|
| 260/280 | 1.97 | 1.98 | 1.75 | 1.70 |
| DNA (ug/ml) | 19.0 | 12.4 | 15.0 | 25.5 |

### Example 8: Extracting Nucleic Acids from EasySep Isolated EVs

Pan-EVs isolated from plasma with an EasySep™ kit (STEMCELL Technologies) were prepared as described in Example 1, and bound to particles as described in Example 3. It was investigated whether or not the presence of particles used in EasySep™-mediated EV enrichment was an impediment to downstream particle-mediated nucleic acid extraction, as described in Example 3.

Table 3 below shows that relative to particle-free EVs, the recovery of DNA was not affected by the presence of particles in the sample prior to nucleic acid extraction.

TABLE 3
Recovery of DNA (in ng/μl, and ng/extraction) from Pan-
EVs with or without particle clean-up. n = 3

| | With particle | Without particle |
|---|---|---|
| Qubit (N = 3) | clean-up | clean-up |
| Avg. DNA/Isolation (ng/ul) | 0.055 | 0.051 |
| Avg. ng DNA/Isolation | 1.80 | 1.64 |

### Example 9: Extracting Nucleic Acids from Organoids

Hepatic organoid (formed using a kit commercialized by STEMCELL Technologies) samples were prepared as described in Example 1, and bound to particles as described in Example 3. RNA was extracted from the samples using either a conventional spin-column (RNeasy, Qiagen) or a one-step particle-based EasySep™ method of this disclosure, as described in Example 3.

In addition, different combinations of lysis buffers (phenol chloroform-based, TRIzol; and a lysis buffer of this disclosure (IH-LB)) and extraction methods (Spin vs EasySep™) were tested.

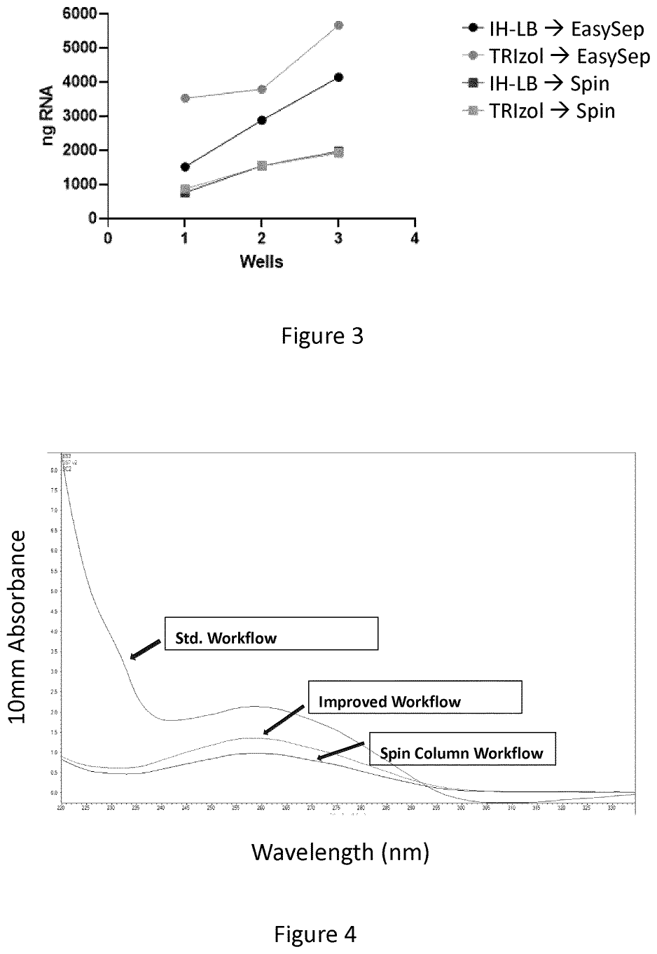

Irrespective of the sample lysis condition, particle-mediated EasySep™ RNA extraction of this disclosure yielded the highest recoveries in comparison to a conventional spin-column approach (FIG. 3, Table 4), while 260/280 purity ratios were consistent across experiments (Table 4).

TABLE 4
Purity and recovery of RNA extracted using different combinations
of lysis conditions and isolation platforms. Recovery values
shown are the average of 3 technical replicates.

NanoDrop values

| Conditions | Average ng RNA/well | 260/280 |
|---|---|---|
| TRIzol → Spin | 759 | 1.93 |
| IH - LB → Spin | 727 | 1.90 |
| IH - LB → EasySep | 1443 | 1.90 |
| TRIzol → EasySep | 2436 | 1.77 |

### Example 10: Extracting Nucleic Acids from a Mouse Liver Tissue

Mouse liver samples were prepared as described in Example 1, and bound to particles as described in Example 3. RNA was extracted from the samples using either a conventional spin-column (RNeasy, Qiagen) (“Spin”) or a one-step particle-based EasySep™ method of this disclosure, as described in Example 3. The same lysis buffers and platform combinations were tested as described in Example 9.

Irrespective of the sample lysis condition, particle-mediated EasySep™ RNA extraction of this disclosure yielded the highest recoveries in comparison to a conventional spin-column approach (Table 5), while 260/280 purity ratios were consistent across experiments (Table 5).

TABLE 5
Purity and recovery of RNA extracted from whole mouse
liver tissue through the method of this disclosure.

NanoDrop values

| Conditions | ng RNA | 260/280 |
|---|---|---|
| TRIzol → Spin | 190,935 | 2.05 |
| IH-LB → Spin | 42,047 | 2.07 |
| IH-LB → EasySep | 221,220 | 1.93 |
| TRIzol → EasySep | 308,993 | 2.02 |

### Example 11: Suitability of the Extracted Nucleic Acids for Downstream Application

Nucleic acids extracted according to a method of this disclosure were tested in downstream RT-qPCR. Extracted RNAs were either treated with or untreated with DNase prior to reverse transcription, and average Ct values of a target gene were comparable in both conditions, irrespective of the amount of input RNA reverse transcribed (Table 6).

Thus, DNase treatment status of RNA extracted using a particle-based EasySep™ nucleic acid extraction platform prior to analysis by RT-qPCR does not negatively influence analysis by RT-qPCR.

TABLE 6
Comparable Ct values of RNA extracted from day-7 organoids
(n = 1) regardless of DNAse treatment status.

| | |
|---|---|
| +DNase | −DNase |

| | |
|---|---|
| ng RNA input | Average Ct |

| | | |
|---|---|---|
| 800 | 26.28 | 26.94 |
| 80 | 29.61 | 30.27 |
| 8 | 32.83 | 33.50 |

### Example 12: Extracting Nucleic Acids from Whole Blood by an Improved Method of this Disclosure

Nucleic acids were extracted from whole blood using either the one-step particle-based method (“Standard Workflow”) or the two-step particle-based method (“Improved Workflow”) as described in Example 3, and compared to a conventional spin column approach.

Spectrophotometric absorption spectra showed that the nucleic acids extracted from a complex sample according to the Improved Workflow was improved, in terms of both recovery and purity, over both the spin column workflow and the Standard Workflow (Table 7). Further, nucleic acids from both the spin-column workflow and the Improved Workflow had less contamination compared to the Standard Workflow (FIG. 4).

Thus, the results show that an improved, two-step particle based method of this disclosure yields optimal purity ratios and highest recoveries for nucleic acids extracted from a complex biological sample.

TABLE 7
Comparison of nucleic acid purity, and DNA and RNA recovery
using a one-step particle-based workflow (Standard Workflow),
an improved two-step particle-based workflow (Improved
Workflow), and a conventional spin column approach (Spin
column Workflow). Values are the mean of 2 technical
replicates of 3 biological replicates.

| | Standard | Improved | Spin Column |
|---|---|---|---|
| | Workflow | Workflow | Workflow |
| Avg 260/280 | 1.74 ± 0.07 | 1.87 ± 0.02 | 1.87 ± 0.05 |
| Avg 260/230 | 0.52 ± 0.10 | 2.28 ± 0.04 | 1.98 ± 0.44 |
| Avg μg DNA | 2.53 ± 0.88 | 2.78 ± 0.47 | 1.97 ± 0.30 |
| Avg μg RNA | 0.28 ± 0.16 | 0.30 ± 0.10 | 0.21 ± 0.06 |

### Example 13: Varying Wash Cycles in a Two-Step Nucleic Acid Extraction Method

A whole blood sample was prepared essentially as described in Example 1. Recapture by second particles of nucleic acids eluted from first particles versus recapturing eluted nucleic acids by the same particles was evaluated in terms of purity and recovery, essentially as described in Example 3.

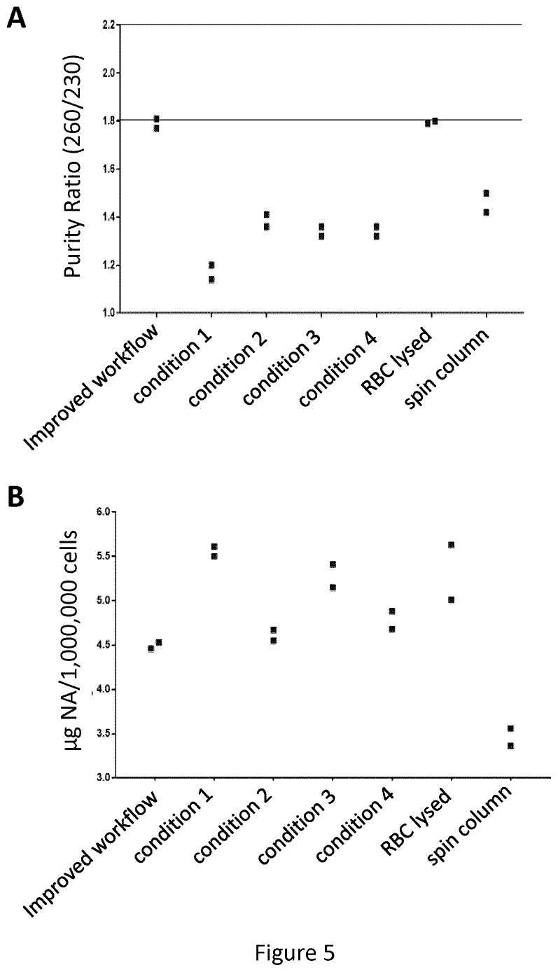

Highest 260/230 purity (FIG. 5A) and comparable recovery (FIG. 5B) were obtained when nucleic acids were recaptured to fresh particles (“Improved Workflow”) in comparison to both re-capturing to the same particles (conditions 1-4) or a spin column method. Addition of fresh particles for the second capture yielded purity and recovery comparable to the RBC lysis method of nucleic acid extraction from whole blood (FIG. 5).

The number and timing of washes on nucleic acid purity and recovery was also explored to see if purity could be enhanced in a modified two-step approach (where nucleic acids are recaptured by the original particles). While a no-wash condition (Condition 1) exhibited highest recoveries, it also resulted in the lowest purity (FIG. 5). Performing one, two, or three washes during the first capture cycle (Conditions 2, 3, and 4, respectively) with three washes during the second cycle of recapture to the same particles only marginally improved purity (FIG. 5A).

Accordingly, all particle-based approaches outperformed spin-column nucleic acid extraction from a complex biological sample (e.g. whole blood) in terms of recovery, and only the no-wash control (condition 1) yielded purities lower than the spin-column extractions (FIG. 5B). Thus, particle-based extractions from whole blood, in accordance with the Examples disclosed herein, are better for nucleic acid extraction from complex samples compared to spin columns. Best results were obtained using fresh particles in each step of a two-step capture and release EasySep™ protocol of this disclosure.

### Example 14: Priming Buffer Required for Nucleic Acids Re-Capture to Second Particles

Nucleic acid recovery was studied either in the presence or in the absence of priming buffer (formulated consistent with a lysis buffer of this disclosure) in the first elution (prior to the second capture), in the methods as described in Examples 12 and 13.

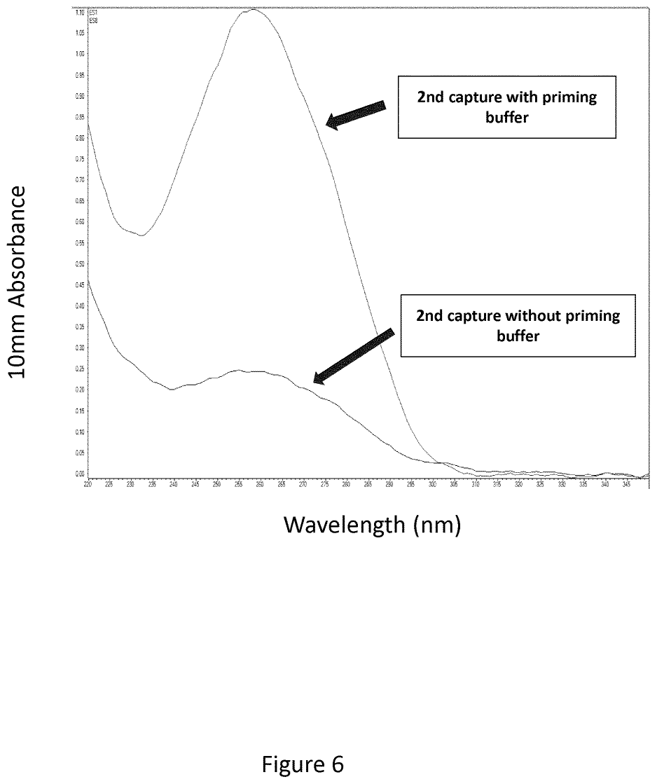

Addition of priming buffer to the first elution resulted in a significant increase in nucleic acid recovery compared to performing the second capture in the absence of priming buffer (FIG. 6).

### Example 15: Purity and Recovery in Spin-Column and Two-Step Nucleic Acid Extractions

Whole blood samples were prepared as described in Example 1, and subjected to column-based nucleic acid extraction (RNEasy) and a one- or two-step particle-based EasySep™ approach (“Standard” or “Improved” workflows), as described in Examples 3, and 12-14.

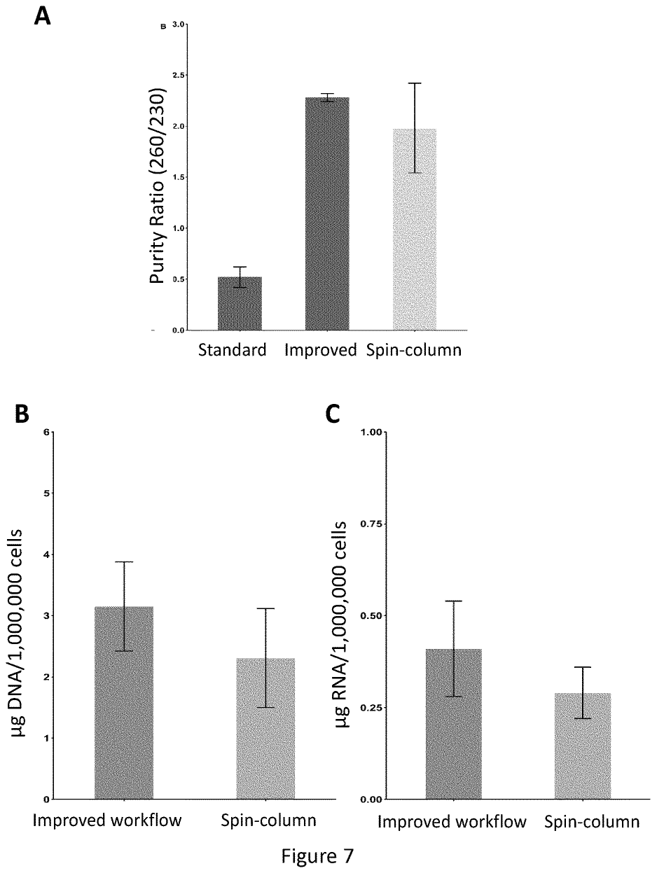

Nucleic acid purity (260/230 ratio) was highest using the Improved Workflow and the column-based workflow (FIG. 7A), with slightly higher purity for the Improved Workflow. Normalized nucleic acid recovery (μg/10⁶ cells) using the Improved Workflow was higher for both DNA (FIG. 7B) and RNA (FIG. 7C) in comparison to recoveries using a spin-column approach.

1. A method for extracting nucleic acids from a biological sample, the method comprising:
i) preparing a sample lysate by treating the biological sample with a lysis buffer;
i) incubating the sample lysate at a temperature at or above room temperature (RT) and below 60° C.;
iii) adding particles to the sample lysate to bind the nucleic acids;
iv) separating the particles and the nucleic acids bound thereto from the sample lysate; and
v) contacting the particles and the nucleic acids bound thereto with an elution buffer to release the nucleic acids from the particles.

2. The method of claim 1, wherein a purity and/or recovery of the released nucleic acids are higher than a purity and/or recovery of the released nucleic acids when the biological sample of step i) is incubated above room temperature compared to at room temperature.

3. The method of claim 1, wherein the pH of the lysis buffer is between about 4 and 10.

4. The method of claim 1, further comprising pre-heating the elution buffer to a temperature above RT and below 60° C.

5. The method of claim 4, wherein the temperature of the elution buffer is between 30° C. and 60° C.

6. The method of claim 4, wherein pre-heating the elution buffer improves purity and/or recovery of the released nucleic acids compared to the purity and/or recovery of the nucleic acids released in room-temperature elution buffer.

7. The method of claim 1, wherein the particles are added to the sample lysate at a concentration
a) above 1 mg/ml of the sample lysate and below 2.2 mg/ml of the sample lysate, and/or
b) between about 1.2 mg/ml of the sample lysate and 2 mg/ml of the sample lysate.

8. (canceled)

9. The method of claim 1, wherein the biological sample is whole blood, suspended cells, isolated cells, PBMCs, liver tissue, extracellular vesicle, leukapheresis product, single cell suspension, organoids, plasma, and virus.

10. The method of claim 1, wherein the biological sample is a complex sample.

11. The method of claim 1, wherein the biological sample is pre-processed or is not pre-processed.

12. The method of claim 1, wherein the biological sample comprises between 10¹ and 10⁹ cells.

13. The method of claim 1, further comprising subjecting the released nucleic acids directly to one or more downstream applications.

14. The method of claim 1, further comprising adding fresh particles to the released nucleic acids to recapture the released nucleic acids.

15. The method of claim 14, further comprising releasing the recaptured nucleic acids.

16. The method of claim 14, wherein the method does not involve washing the particles and the nucleic acids bound thereto before contacting them with the elution buffer.

17. The method of claim 14, wherein a purity and/or recovery of the recaptured nucleic acids released from the fresh particles is higher than a purity and/or recovery of nucleic acids released in step v).

18. The method of claim 1, further comprising fractionating the particles and a supernatant comprising the released nucleic acids by aspiration or pour-off.

19. The method of claim 1, wherein the particles are silica-based.

20. The method of claim 1, wherein the method elapses between 30-45 minutes.

21. The method of claim 1, wherein the particles are magnetic particles, and the magnetic particles and the nucleic acids bound thereto are separated from the sample lysate by applying a magnetic field to the sample lysate.
